(19) United States
(12) Patent Application Publication
(10) Pub. No.: US 2026/0061367 A1
(43) Pub. Date: Mar. 5, 2026

(54) PHOTOCHEMICALLY CONTROLLED DIRECT AIR CAPTURE OF CARBON DIOXIDE WITH A GUANIDINE PHOTOBASE

(71) Applicant: UT-Battelle, LLC, Oak Ridge, TN (US)

(72) Inventors: Radu Custelcean, Knoxville, TN (US); Jeffrey Einkauf, Knoxville, TN (US)

(21) Appl. No.: 19/317,247

(22) Filed: Sep. 3, 2025

Related U.S. Application Data
(60) Provisional application No. 63/690,375, filed on Sep. 4, 2024.

(51) Int. Cl. B01D 53/78; B01D 53/14; B01D 53/62; B01D 53/96; C07D 213/53; C07D 213/61; C07D 213/65; C07D 213/85

(52) U.S. Cl. CPC B01D 53/78; B01D 53/1425; B01D 53/1475; B01D 53/1493; B01D 53/62; B01D 53/96; C07D 213/53; C07D 213/61; C07D 213/65; C07D 213/85; B01D 2252/20494; B01D 2257/504; B01D 2258/06; B01D 2259/802; B01D 2259/804

(57) ABSTRACT

A compound having the following structure:

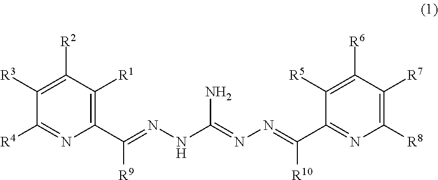

wherein: R¹, R², R³, R⁴, R⁵, R⁶, R⁷, R⁸, R⁹, and R¹⁰ are independently selected from (i) hydrogen atom (H), (ii) alkyl groups (R) containing 1-3 carbon atoms and optionally substituted with one or more fluorine atoms, (iii) —(CH₂)ₙ(OCH₂CH₂)ₘX groups, (iv) OR′ groups, (v) NR′₂ groups, (vi) NHC(O)R′, (vii) C(O)R′ groups, (viii) halogen atoms, (ix) NO₂ groups, and (x) CN groups, wherein R′ groups are independently selected from hydrogen atom (H); alkyl groups (R) containing 1-3 carbon atoms; and groups of the formula —(CH₂)ₙ(OCH₂CH₂)ₘX, wherein n is 0 or an integer of 1-3, m is 0 or an integer of 1-12, and X is H, OH, OCH₃, or OCH₂CH₃. Also described herein are methods for selectively capturing carbon dioxide (CO₂) from a liquid source by use of the above compound.

### CROSS REFERENCE TO RELATED APPLICATION

The present application claims benefit of U.S. Provisional Application No. 63/690,375, filed on Sep. 4, 2024, all of the contents of which are incorporated herein by reference.

### STATEMENT REGARDING FEDERALLY SPONSORED RESEARCH

This invention was made with government support under Prime Contract No. DE-AC05-000R22725 awarded by the U.S. Department of Energy. The government has certain rights in the invention.

### FIELD OF THE INVENTION

The present invention generally relates to compositions and methods for capturing carbon dioxide from liquid solutions, and more particularly, to compounds that function as a photobase and methods for using them in conjunction with a reactive amino acid for selectively capturing and releasing carbon dioxide in aqueous solution.

### BACKGROUND

Direct air capture (DAC) is considered an important element in the effort to reduce atmospheric CO₂, yet the high energies and temperatures involved in current DAC technologies make their large-scale deployment largely impractical and economically unfeasible. In the case of aqueous-based CO₂ absorbents, which have been identified as the basis for one of the most promising and scalable DAC approaches, a large energetic penalty is associated with heating and boiling off water, as required for solvent regeneration in a conventional thermal-swing DAC process. Efforts have been made to reduce this energy penalty by employing alternative solvent regeneration methods based on electrochemical or photochemical pH swings. However, such efforts have been largely unsuccessful.

Moreover, the relatively low concentration of carbon dioxide in the atmosphere, and the strong competition from water vapor, necessitate sorbents or solvents with strong CO₂ binding for efficient direct air capture (DAC). Such strong binding is mostly achieved by chemisorption, typically involving amine- or hydroxide-based sorbents that react with CO₂ and convert it into carbamate or bicarbonate species.

As well known, atmospheric carbon dioxide capture involves acid-base chemistry, as CO₂ is an acidic gas that generates carbonic acid when dissolved in water, thereby requiring a base, such as an amine, to neutralize it. For instance, as well known, glycine, as an aqueous sodium or potassium salt, effectively removes CO₂ from air by converting it into bicarbonate anion via a carbamate intermediate. The reaction is thermodynamically favorable, nominally driven by the efficient, downhill proton transfer from carbonic acid (apparent pKa 6.35) to glycine (pKa 9.58). As a result, reversing this reaction to release the CO₂ and regenerate the glycine amino acid requires back proton transfer against the pKa gradient, from the stronger base glycine to the weaker base bicarbonate, a process that takes energy in the form of heat. Furthermore, in addition to the endothermic CO₂-release reaction, more energy is wasted on heating and boiling off water in the process of regenerating the aqueous amino acid solvent. Chemical degradation of the amino acid may also occur from extensive heating. Thus, there would be a significant benefit in a method that reduces or diminishes the regeneration energy penalty discussed above while also preventing degradation of the amino acid sorbent.

### SUMMARY

The present disclosure foremost describes a process for capturing carbon dioxide by use of a photobase compound in conjunction with an amino acid, wherein the method advantageously permits easy regeneration of the photobase and amino acid with a highly diminished energy penalty. In the method, carbon dioxide from the atmosphere or process stream is captured with an aqueous solution containing a bis-pyridine guanidine photobase and a small peptide, using an ultraviolet or visible light stimulus, and subsequently released when the light stimulus is removed. In this approach, the bis-pyridine guanidine photobase becomes more basic after exposure to the light stimulus. The pH swing of this photobase during photoirradiation facilitates a proton transfer event, which activates a small peptide for CO₂ capture. Removing the solution from UV light initiates the release of CO₂. The process can advantageously be cycled between capture and release states multiple times with minimal loss in efficiency.

More particularly, the method includes the following steps:
(i) providing an aqueous solution containing (a) an aqueous solvent, (b) an amino acid or oligopeptide of the formula H₃N⁺-L-COO⁻, wherein L has the formula —(CH₂)ᵣ— wherein r is an integer of 1-6 with optional interruption of one or more carbon-carbon bonds with one or more —C(O)NH— linkages, and (c) a photoactive (diiminoguanidine) compound of the following Formula (1);
(ii) contacting a source of CO₂ with the aqueous solution during exposure of the aqueous solution to electromagnetic radiation (hv) having a wavelength in a range of 200-600 nm to convert the E,E (open) configuration of compound of Formula (1) to a Z,Z (closed) configuration of Formula (1′), wherein the Z,Z configuration is more basic than the E,E configuration and has a higher pKa than the amino acid or oligopeptide in order to abstract an ammonium hydrogen atom from the amino acid or oligopeptide, wherein the deprotonated amino acid or oligopeptide captures carbon dioxide; and
(iii) ceasing exposure to the electromagnetic radiation which results in conversion of the Z,Z form back to the E,E form with simultaneous protonation of the bicarbonate (HCO₃⁻) anion and release of CO₂.

In some embodiments, the photoactive (diiminoguanidine) compound has the following structure:

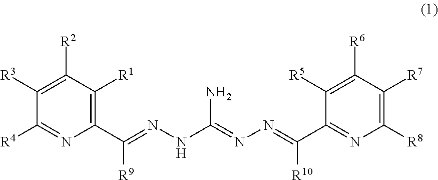

wherein: R¹, R², R³, R⁴, R⁵, R⁶, R⁷, R⁸, R⁹, and R¹⁰ are independently selected from (i) hydrogen atom (H), (ii) alkyl groups (R) containing 1-3 carbon atoms and optionally substituted with one or more fluorine atoms, (iii) —(CH₂)ₙ(OCH₂CH₂)ₘX groups, (iv) OR′ groups, (v) NR′₂ groups, (vi) NHC(O)R′, (vii) C(O)R′ groups, (viii) halogen atoms, (ix) NO₂ groups, and (x) CN groups, wherein R′ groups are independently selected from hydrogen atom (H); alkyl groups (R) containing 1-3 carbon atoms; and groups of the formula —(CH₂)ₙ(OCH₂CH₂)ₘX, wherein n is 0 or an integer of 1-3, m is 0 or an integer of 1-12, and X is H, OH, OCH₃, or OCH₂CH₃; provided that n and m are not both 0; n is not 0 for R′ in OR′ and NR′₂; and if m is 0, then X is OH, OCH₃, or OCH₂CH₃.

In some embodiments of Formula (1), a proviso is included that at least one of R₁, R², R³, R⁴, R⁵, R⁶, R⁷, R⁸, R⁹, and R¹⁰ is independently selected from NO₂, CN, —(CH₂)ₙ(OCH₂CH₂)ₘX, OR′, NR′₂, NHC(O)R′, and C(O)R′, wherein at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′ and NR′₂; and if m is 0, then X is OH, OCH₃, or OCH₂CH₃. The foregoing proviso captures photoactive compounds that are highly water or aqueous soluble.

In some embodiments, precisely or at least one or two of R¹, R², R³, R⁴, R⁵, R⁶, R⁷, R¹, R⁹, and R¹⁰ (or precisely or at least one or two of R¹, R², R³, R⁴, R⁵, R⁶, R⁷, and R⁸ or precisely or at least one or two of R², R³, R⁶, and R⁷ or one or both of R³ and R⁷) is/are independently selected from NO₂, CN, —(CH₂)ₙ(OCH₂CH₂)ₘX, OR′, NR′₂, NHC(O)R′, and C(O)R′, wherein R′ is independently selected from hydrogen atom (H); alkyl groups (R) containing 1-3 carbon atoms; and groups of the formula —(CH₂)ₙ(OCH₂CH₂)ₘX, wherein n is 0 or an integer of 1-3, m is 0 or an integer of 1-12, and X is H, OH, OCH₃, or OCH₂CH₃; provided that n and m are not both 0; n is not 0 for R′ in OR′ and NR′₂; and if m is 0, then X is OH, OCH₃, or OCH₂CH₃. In some embodiments, at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′ and NR′₂; and if m is 0, then X is OH, OCH₃, or OCH₂CH₃.

In some embodiments, the compound has the following structure:

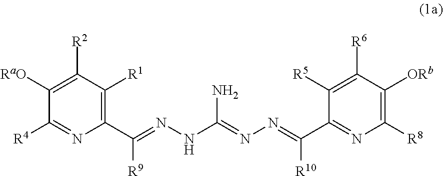

wherein at least one R¹, R², R⁴, R⁵, R⁶, R⁸, R⁹, and R¹⁰ are as defined above, and R^{a} and R^{b} are selected from H, alkyl groups (R) containing 1-3 carbon atoms, and groups of the formula —(CH₂)ₙ(OCH₂CH₂)ₘX, wherein n is an integer of 1-3, m is 0 or an integer of 1-12, and X is H, OH, OCH₃, or OCH₂CH₃, provided that if m is 0, then X is OH, OCH₃, or OCH₂CH₃.

In some embodiments, precisely or at least one or two of R¹, R², R³, R⁴, R⁵, R⁶, R⁷, and R¹ (or precisely or at least one or two of R², R³, R⁶, and R⁷ or one or both of R³ and R⁷) is/are NO₂. In other embodiments, precisely or at least one or two of R¹, R², R³, R⁴, R⁵, R⁶, R⁷, and R⁸ (or precisely or at least one or two of R², R³, R⁶, and R⁷ or one or both of R³ and R⁷) is/are CN. In other embodiments, precisely or at least one or two of R¹, R², R³, R⁴, R⁵, R⁶, R⁷, and R⁸ (or precisely or at least one or two of R², R³, R⁶, and R⁷ or one or both of R³ and R⁷) is/are OR′ wherein R′ is as defined above. In other embodiments, precisely or at least one or two of R¹, R², R³, R⁴, R⁵, R⁶, R⁷, and R⁸ (or precisely or at least one or two of R², R³, R⁶, and R⁷ or one or both of R³ and R⁷) is/are halogen atom (e.g., F, Cl, or Br atom).

The CO₂ capture and release process described above can be conveniently depicted by the following reaction scheme:

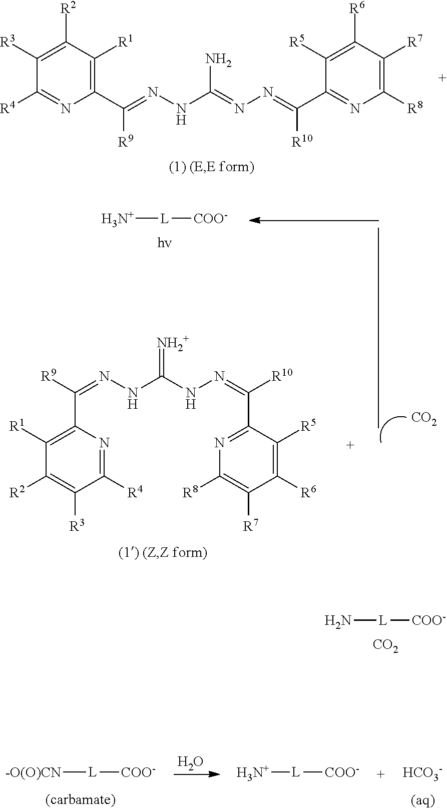

In more particular embodiments, the present disclosure is directed to a photochemically-driven DAC process (photo-DAC) in which atmospheric CO₂ capture by an aqueous oligopeptide, e.g., glycylglycine (GlyGly), is achieved by a pH swing using a pyridine-substituted diaminoguanidine such as within the scope of Formula (1) above, e.g., PyDIG, as shown in the above scheme. Upon irradiation with UV light, the PyDIG photobase undergoes photoisomerization from the E,E to the Z,Z isomer, corresponding to a pKa increase of 2.8 units that activates GlyGly for DAC through deprotonation. After the GlyGly/PyDIG solvent is saturated with atmospheric carbon dioxide, leaving it in the dark under ambient conditions leads to isomerization of PyDIG from the Z,Z back to the E,E isomer, which is accompanied by a pH drop and CO₂ release. To demonstrate the recyclability of the GlyGly/PyDIG solvent, seven consecutive DAC cycles were completed, with a measured average cyclic capacity in the range of 0.21-0.26 mols CO₂ per mol of GlyGly/PyDIG, and minimal loss in efficiency from one cycle to another. These results open the prospect for energy-efficient DAC cycles completed entirely at ambient conditions, thereby avoiding the significant energy penalties associated with heating and boiling aqueous solvents.

The guanidine photobase prototype, bis-2-pyridyldiiminoguanidine (PyDIG, FIG. 2) and other diaminoguanidine compounds within the scope of Formula (1) can be easily prepared with economical starting materials and minimal workup. PyDIG, in particular, undergoes reversible photoisomerization between the neutral form (E,E-PyDIG) to the protonated, basic, closed form (Z,Z-PyDIGH⁺) when exposed to UV light for 24 h in water. By adding a small peptide, glycylglycine (GlyGly), the method can achieve capture of CO₂ from air by leveraging the change in pKa from 6.31 in the open form to 9.72 in the closed form of PyDIG by light irradiation, which facilitates proton transfer from the zwitterionic GlyGly peptide to the photobase. This, in turn, activates the GlyGly peptide for atmospheric CO₂ capture, through a carbamate intermediate, all the way to bicarbonate anions. Furthermore, the change in PyDIG basicity promotes an overall increase in the pH of the solution by production of hydroxide ions that can also capture CO₂. This synergistic process between the PyDIG photobase and the GlyGly peptide results in an overall capture of 0.421±0.021 mM CO₂ for every 1 mM of PyDIG and GlyGly. The whole process can be reversed by stopping exposure to the UV or visible light, which triggers the conversion of the closed Z,Z-PyDIGH⁺ form back to the open E,E-PyDIG form. This dark reaction releases the proton back into solution, leading to protonation of the bicarbonate anion and release of the CO₂ (ca. 70% of the captured CO₂ is released). The CO₂ capture and release cycle can be advantageously repeated multiple times with minimal loss in efficiency.

### BRIEF DESCRIPTION OF THE DRAWINGS

FIG. 1. Schematic showing photo-DAC process using a photobase (PB), as demonstrated in this study. Photoexcitaton and isomerization of the PB (right) raises the solution pH, which activates the sorbent (S) via deprotonation. Subsequently, S captures atmospheric CO₂ by converting it to bicarbonate. Moving the PB solution into the dark (left) allows the excited PB to undergo thermal relaxation, which lowers the solution pH and converts the HCO₃⁻ back to CO₂, which is released.

FIG. 2. Schematic showing a photo-DAC cycle using aqueous PyDIG photobase and diglycine (GlyGly) oligopeptide.

FIG. 3. Schematic showing photochemical modulation of PyDIG basicity and solution alkalinity.

FIGS. 4a-4b. FIG. 4a is a graph showing evolution of the pH of a 1 mM PyDIG and GlyGly solution during 2 h of UV irradiation, followed by 24 h of atmospheric CO₂ capture, and 5 d of thermal relaxation in the dark; FIG. 4b is a graph showing variation of the concentration of the atmospheric CO2 absorbed in the PyDIG/GlyGly solution, measured by total inorganic carbonate (TIC) for three independent cycling experiments, for consecutive DAC cycles alternating photoirradiations (dashed or dotted regions) and thermal relaxation at rt in the dark (white). Experiment set 1 was phoroirradiated for 24 h while experiments 2 and 3 were photoirradiated for 2 h.

### DETAILED DESCRIPTION

In one aspect, the present disclosure is directed to specialized photoactive compounds that have an ability to change in configuration along with increase in hydrogen abstraction ability upon exposure to ultraviolet or visible electromagnetic radiation, typically in a range of 200-600 nm. The increase in hydrogen abstraction ability should be sufficient to abstract an amine-bound proton from an N-protonated amino acid. The photoactive compounds described herein having this ability are within the class of diiminoguanidine-dipyridyl compounds, as further described below.

The diiminoguanidine-dipyridyl photoactive compounds are within the scope of the following generic structure:

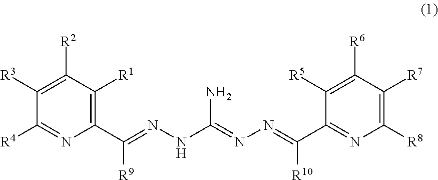

In Formula (1) above, R¹, R², R³, R⁴, R⁵, R⁶, R⁷, R⁸, R⁹, and R¹⁰ are independently selected from (i) hydrogen atom (H), (ii) alkyl groups (R) containing 1-3 carbon atoms and optionally substituted with one or more fluorine atoms, (iii) —(CH₂)ₙ(OCH₂CH₂)ₘX groups, (iv) OR′ groups, (v) NR′₂ groups, (vi) NHC(O)R′, (vii) C(O)R′ groups, (viii) halogen atoms, (ix) NO₂ groups, and (x) CN groups, wherein R′ groups are independently selected from hydrogen atom (H); alkyl groups (R) containing 1-3 carbon atoms; and groups of the formula —(CH₂)ₙ(OCH₂CH₂)ₘX, wherein n is 0 or an integer of 1-3, m is 0 or an integer of 1-12, and X is H, OH, OCH₃, or OCH₂CH₃; provided that n and m are not both 0; n is not 0 for R′ in OR′ and NR′₂; and if m is 0, then X is OH, OCH₃, or OCH₂CH₃. In some embodiments, one or both of R⁹ and R¹⁰ are hydrogen atoms. In other embodiments, one or both of R⁹ and R¹⁰ are not hydrogen atoms, or more particularly, selected from any of the groups (ii)-(x) provided above.

In some embodiments, one or more of (or all of) R¹, R², R³, R⁴, R⁵, R⁶, R⁷, R⁸, R⁹, and R¹⁰ are hydrogen atoms. In other embodiments, precisely or at least one, two, three, four, or more of (or all of) R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) are not hydrogen atoms, or more particularly, they are selected from any of the groups (ii), (iii), (iv), (v), (vi), (vii), (viii), (ix), or (x) provided above, wherein the selected groups may be the same or different.

In a first set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is an alkyl group (R) group containing 1-3 carbon atoms, optionally substituted with one or more fluorine atoms. The alkyl group may be, for example, methyl, ethyl, n-propyl, isopropyl, fluoromethyl, difluoromethyl, trifluoromethyl, perfluoroethyl, or perfluoropropyl. If more than one R group is present, the R groups may be the same or different. In some embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) are selected from any one or more of the R groups provided above and hydrogen atoms, and notably, one or more additional groups selected from groups (iii)-(x) may or may not be present. In some embodiments, precisely or at least one of R¹, R², R³, and R⁴ is an R group and/or precisely or at least one of R⁵, R⁶, R⁷, and R⁸ is an R group, wherein remaining groups may be H atoms. In particular embodiments, one or both of R³ and R⁷ are R groups, and/or one or both of R² and R⁶ are R groups, wherein remaining groups may be H atoms. In another embodiment, none of R¹, R², R³, R⁴, R⁵, R⁶, R⁷, R⁸, R⁹, and R¹⁰ or none of R¹, R², R³, R⁴, R⁵, R⁶, R⁷, and R⁸ are R groups.

In a second set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from —(CH₂)ₙ(OCH₂CH₂)ₘX groups, wherein n is 0 or an integer of 1, 2, or 3, m is 0 or an integer of 1-12, and X is H, OH, OCH₃, or OCH₂CH₃. In the case where m is 0, then X is OH, OCH₃, or OCH₂CH₃ (i.e., X is not H in that instance). In some embodiments, n and m may both be 0, in which case the —(CH₂)ₙ(OCH₂CH₂)ₘX group simplifies to an X group. In other embodiments, n and m are not both 0. For example, n may be 1, 2, or 3 while m is 0, or n may be 0 while m is 1, 2, 3, 4, 5, 6, 7, 8, 9, or 10 or within any range therein (e.g., 1-12, 2-10, 3-10, 4-10, 1-8, 2-8, 3-8, 4-8, 1-6, 2-6, 3-6, or 4-6). In some embodiments, n and m are both not zero, e.g., n may be 1, 2, or 3 while m is 1, 2, 3, 4, 5, 6, 7, 8, 9, or 10 or within any range therein (e.g., 1-12, 2-10, 3-10, 4-10, 1-8, 2-8, 3-8, 4-8, 1-6, 2-6, 3-6, or 4-6). If more than one —(CH₂)ₙ(OCH₂CH₂)ₘX group is present, the groups may be the same or different (i.e., may contain the same or different n, m, and X variables). In some embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from —(CH₂)ₙ—(OCH₂CH₂)ₘX groups (as defined above) and hydrogen atoms, and notably, one or more additional groups selected from groups (ii) and (iv)-(x) may or may not be present. In some embodiments, precisely or at least one of R¹, R², R³, and R⁴ is an —(CH₂)ₙ—(OCH₂CH₂)ₘX group and/or precisely or at least one of R⁵, R⁶, R⁷, and R⁸ is an —(CH₂)ₙ(OCH₂CH₂)ₘX group, wherein remaining groups may be H atoms. In particular embodiments, one or both of R³ and R⁷ are —(CH₂)ₙ(OCH₂CH₂)ₘX groups, and/or one or both of R² and R⁶ are —(CH₂)ₙ(OCH₂CH₂)ₘX groups, wherein remaining groups may be H atoms. In another embodiment, none of R¹, R², R³, R⁴, R⁵, R⁶, R⁷, R⁸, R⁹, and R¹⁰ or none of R¹, R², R³, R⁴, R⁵, R⁶, R⁷, and R⁸ are —(CH₂)ₙ(OCH₂CH₂)ₘX groups.

In a third set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from OR′ groups, wherein R′ groups are independently selected from hydrogen atom (H); alkyl groups (R) containing 1-3 carbon atoms; and groups of the formula —(CH₂)ₙ—(OCH₂CH₂)ₘX, with variables n, m, and X as provided above, except that n is not 0 for R′ in OR′ (i.e., n is an integer of 1-3 in this instance). When R′ is H, then OR′ is OH. When R′ is R, then OR′ is OR, such a methoxy, ethoxy, n-propoxy, or isopropoxy group. If more than one OR′ group is present, the groups may be the same or different (i.e., may contain the same or different R′ groups). In some embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from OR′ groups (as defined above) and hydrogen atoms, and notably, one or more additional groups selected from groups (ii), (iii), and (v)-(x) may or may not be present. In some embodiments, precisely or at least one of R¹, R², R³, and R⁴ is an OR′ group and/or precisely or at least one of R⁵, R⁶, R⁷, and R⁸ is an OR′ group, wherein remaining groups may be H atoms. In particular embodiments, one or both of R³ and R⁷ are OR′ groups, and/or one or both of R² and R⁶ are OR′ groups, wherein remaining groups may be H atoms. For any of the possibilities provided above, in some embodiments, at least one (or all) of the OR′ groups contain an R′ group that is a group of the formula —(CH₂)ₙ(OCH₂CH₂)ₘX, with variables n, m, and X as provided above, except that n is not 0 for R′ in OR′ (i.e., n is an integer of 1, 2, or 3 in this instance). In some embodiments, OR′ corresponds to a group of the formula —OCH₂CH₂OH, —OCH₂CH₂OCH₂CH₂OH, —O(CH₂CH₂O)₂CH₂CH₂OH, or —O(CH₂CH₂O)₃CH₂CH₂OH. In some embodiments, one or both of R³ and R⁷ and/or one or both of R² and R⁶ are any of the foregoing specific groups, wherein remaining groups may be H atoms. In another embodiment, none of R¹, R², R³, R⁴, R⁵, R⁶, R⁷, R⁸, R⁹, and R¹⁰ or none of R¹, R², R³, R⁴, R⁵, R⁶, R⁷, and R⁸ are OR′ groups, or alternatively, none of the foregoing groups may be an OH group or OR group.

In a fourth set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from NR′₂ groups, wherein R′ groups are independently selected from hydrogen atom (H); alkyl groups (R) containing 1-3 carbon atoms; and groups of the formula —(CH₂)ₙ(OCH₂CH₂)ₘX, with variables n, m, and X as provided above, except that n is not 0 for R′ in OR′ (i.e., n is an integer of 1-3 in this instance). When both R′ are H, then NR′₂ is NH₂. When one R′ is H and the other is R, then NR′₂ is NHR, such methylamine or ethylamine. When both R′ are R, then NR′₂ is NR₂, such as dimethylamine or diethylamine group. If more than one NR′₂ group is present, the groups may be the same or different (i.e., may contain the same or different R′ groups). In some embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from NR′₂ groups (as defined above) and hydrogen atoms, and notably, one or more additional groups selected from groups (ii)-(iv) and (vi)-(x) may or may not be present. In some embodiments, precisely or at least one of R¹, R², R³, and R⁴ is an NR′₂ group and/or precisely or at least one of R⁵, R⁶, R⁷, and R⁸ is an NR′₂ group, wherein remaining groups may be H atoms. In particular embodiments, one or both of R³ and R⁷ are NR′₂ groups, and/or one or both of R² and R⁶ are NR′₂ groups, wherein remaining groups may be H atoms. For any of the possibilities provided above, in some embodiments, at least one (or all) of the NR′₂ groups contain one or both R′ groups that is/are a group of the formula —(CH₂)ₙ(OCH₂CH₂)ₘX, with variables n, m, and X as provided above, except that n is not 0 for R′ in NR′₂ (i.e., n is an integer of 1-3 in this instance). In another embodiment, none of R¹, R², R³, R⁴, R⁵, R⁶, R⁷, R⁸, R⁹, and R¹⁰ or none of R¹, R², R³, R⁴, R⁵, R⁶, R⁷, and R⁸ are NR′₂ groups, or alternatively, none of the foregoing groups may be an NH₂, NHR, or NR₂ group.

In a fifth set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from NHC(O)R′ groups, wherein R′ groups are independently selected from hydrogen atom (H); alkyl groups (R) containing 1-3 carbon atoms; and groups of the formula —(CH₂)ₙ(OCH₂CH₂)ₘX, with variables n, m, and X as provided earlier above. When n is 1, the NHC(O)R′ group is an amide group. When n is 0, the NHC(O)R′ group is a carbamate group. If more than one NHC(O)R′ group is present, the groups may be the same or different (i.e., may contain the same or different R′ groups). In some embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from NHC(O)R′ groups (as defined above) and hydrogen atoms, and notably, one or more additional groups selected from groups (ii)-(v) and (vii)-(x) may or may not be present. In some embodiments, precisely or at least one of R¹, R², R³, and R⁴ is a NHC(O)R′ group and/or precisely or at least one of R⁵, R⁶, R⁷, and R⁸ is an NHC(O)R′ group, wherein remaining groups may be H atoms. In particular embodiments, one or both of R³ and R⁷ are NHC(O)R′ groups, and/or one or both of R² and R₆ are NHC(O)R′ groups, wherein remaining groups may be H atoms. For any of the possibilities provided above, in some embodiments, at least one (or all) of the NHC(O)R′ groups select R′ as a group of the formula —(CH₂)ₙ(OCH₂CH₂)ₘX, with variables n, m, and X as provided earlier above. In another embodiment, none of R¹, R², R³, R⁴, R⁵, R⁶, R⁷, R⁸, R⁹, and R¹⁰ or none of R¹, R², R³, R⁴, R⁵, R⁶, R⁷, and R⁸ are NHC(O)R′ groups, or alternatively, none of the foregoing groups may be an NHC(O)H or NHC(O)R group.

In a sixth set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from C(O)R′ groups, wherein R′ groups are independently selected from hydrogen atom (H); alkyl groups (R) containing 1-3 carbon atoms; and groups of the formula —(CH₂)ₙ(OCH₂CH₂)ₘX, with variables n, m, and X as provided earlier above. When n is 1, the C(O)R′ group is ketone group. When n is 0, the C(O)R′ group is an ester group. When R′ is a hydrogen (H) atom, the —C(O)R′ group is an aldehyde (—C(O)H) group; when R′ is an R group, the —C(O)R′ group is a ketone (—C(O)R) group. If more than one C(O)R′ group is present, the groups may be the same or different (i.e., may contain the same or different R′ groups). In some embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from C(O)R′ groups (as defined above) and hydrogen atoms, and notably, one or more additional groups selected from groups (ii)-(vi) and (viii)-(x) may or may not be present. In some embodiments, precisely or at least one of R¹, R², R³, and R⁴ is a C(O)R′ group and/or precisely or at least one of R⁵, R⁶, R⁷, and R⁸ is a C(O)R′ group, wherein remaining groups may be H atoms. In particular embodiments, one or both of R³ and R⁷ are C(O)R′ groups, and/or one or both of R² and R⁶ are C(O)R′ groups, wherein remaining groups may be H atoms. For any of the possibilities provided above, in some embodiments, at least one (or all) of the C(O)R′ groups select R′ as a group of the formula —(CH₂)ₙ(OCH₂CH₂)ₘX, with variables n, m, and X as provided earlier above. In another embodiment, none of R¹, R², R³, R⁴, R⁵, R⁶, R⁷, R⁸, R⁹, and R¹⁰ or none of R¹, R², R³, R⁴, R⁵, R⁶, R⁷, and R⁸ are C(O)R′ groups, or alternatively, none of the foregoing groups may be a C(O)H or C(O)R group.

In a seventh set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are halogen atom(s), or precisely or at least three or four of R¹, R², R³, R⁴, R⁵, R⁶, R⁷, R⁸, R⁹, and R¹⁰ (or precisely or at least three or four of R¹, R², R³, R⁴, R⁵, R⁶, R⁷, and R⁸) is/are halogen atom(s). The halogen atom(s) may be selected from fluorine, chlorine, bromine, and iodine atoms. If more than one halogen atom is present, the halogen atoms may be the same or different. In some embodiments, R¹, R², R³, R⁴, R⁵, R⁶, R⁷, R⁸, R⁹, and R¹⁰ (or R¹, R², R³, R⁴, R⁵, R⁶, R⁷, and R⁸) are selected from halogen atoms and hydrogen atoms, and one or more additional groups selected from groups (ii)-(vii), (ix), and (x) may or may not be present. In some embodiments, precisely or at least one of R¹, R², R³, and R⁴ is a halogen atom and/or precisely or at least one of R⁵, R⁶, R⁷, and R⁸ is a halogen atom, wherein remaining groups may be H atoms. In particular embodiments, one or both of R³ and R⁷ are halogen atoms, or one or both of R² and R⁶ are halogen atoms, wherein remaining groups may be H atoms.

In an eighth set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are NO₂ group(s), or precisely or at least three or four of R¹, R², R³, R⁴, R⁵, R⁶, R⁷, R⁸, R⁹, and R¹⁰ (or precisely or at least three or four of R¹, R², R³, R⁴, R⁵, R⁶, R⁷, and R⁸) is/are NO₂ group(s). In some embodiments, R¹, R², R³, R⁴, R⁵, R⁶, R⁷, R⁸, R⁹, and R¹⁰ (or R¹, R², R³, R⁴, R⁵, R⁶, R⁷, and R⁸) are selected from NO₂ group(s) and hydrogen atoms, and one or more additional groups selected from groups (ii)-(viii) and (x) may or may not be present. In some embodiments, precisely or at least one of R¹, R², R³, and R⁴ is a NO₂ group and/or precisely or at least one of R⁵, R⁶, R⁷, and R⁸ is a NO₂ group, wherein remaining groups may be H atoms. In particular embodiments, one or both of R³ and R⁷ are NO₂ groups, or one or both of R² and R₆ are NO₂ groups, wherein remaining groups may be H atoms. In another embodiment, none of R¹, R², R³, R⁴, R⁵, R⁶, R⁷, R⁸, R⁹, and R¹⁰ or none of R¹, R², R³, R⁴, R⁵, R⁶, R⁷, and R⁸ are NO₂ groups.

In a ninth set of embodiments, precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are CN group(s), or precisely or at least three or four of R¹, R², R³, R⁴, R⁵, R⁶, R⁷, R⁸, R⁹, and R¹⁰ (or precisely or at least three or four of R¹, R², R³, R⁴, R⁵, R⁶, R⁷, and R⁸) is/are CN group(s). In some embodiments, R¹, R², R³, R⁴, R⁵, R⁶, R⁷, R⁸, R⁹, and R¹⁰ (or R¹, R², R³, R⁴, R⁵, R⁶, R⁷, and R¹) are selected from CN group(s) and hydrogen atoms, and one or more additional groups selected from groups (ii)-(ix) may or may not be present. In some embodiments, precisely or at least one of R¹, R², R³, and R⁴ is a CN group and/or precisely or at least one of R⁵, R⁶, R⁷, and R⁸ is a CN group, wherein remaining groups may be H atoms. In particular embodiments, one or both of R³ and R⁷ are CN groups, or one or both of R² and R⁶ are CN groups, wherein remaining groups may be H atoms. In another embodiment, none of R¹, R², R³, R⁴, R⁵, R⁶, R⁷, R⁸, R⁹, and R¹⁰ or none of R¹, R², R³, R⁴, R⁵, R⁶, R⁷, and R⁸ are CN groups.

In some embodiments of Formula (1), a proviso is included that precisely or at least one or two of R¹, R², R³, R⁴, R⁵, R⁶, R⁷, R⁸, R⁹, and R¹⁰ (or precisely or at least one or two of R¹, R², R³, R⁴, R⁵, R⁶, R⁷, and R⁸ or precisely or at least one or two of R², R³, R⁶, and R⁷) is/are independently selected from NO₂, CN, —(CH₂)ₙ(OCH₂CH₂)ₘX, OR′, NR′₂, NHC(O)R′, and C(O)R′, or any sub-grouping of these, wherein, in some embodiments, at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃. In some embodiments, n and m are not both 0. The variable n is not 0 for R′ in OR′ and NR′₂; and if m is 0, then X is OH, OCH₃, or OCH₂CH₃. The foregoing proviso captures photoactive compounds that are highly water or aqueous soluble.

In particular embodiments, the photoactive compound has the following structure:

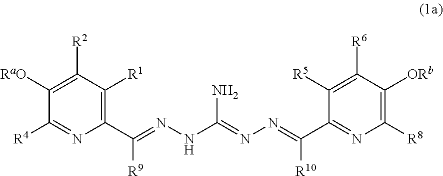

wherein at least one R¹, R², R⁴, R⁵, R⁶, R⁸, R⁹, and R¹⁰ is/are as defined above, and R^{a} and R^{b} are selected from R groups or —(CH₂)ₙ(OCH₂CH₂)ₘX, wherein n is an integer of 1-3, m is 0 or an integer of 1-12, and X is H, OH, OCH₃, or OCH₂CH₃, provided that if m is 0, then X is OH, OCH₃, or OCH₂CH₃.

Some examples of specific compounds include the following:

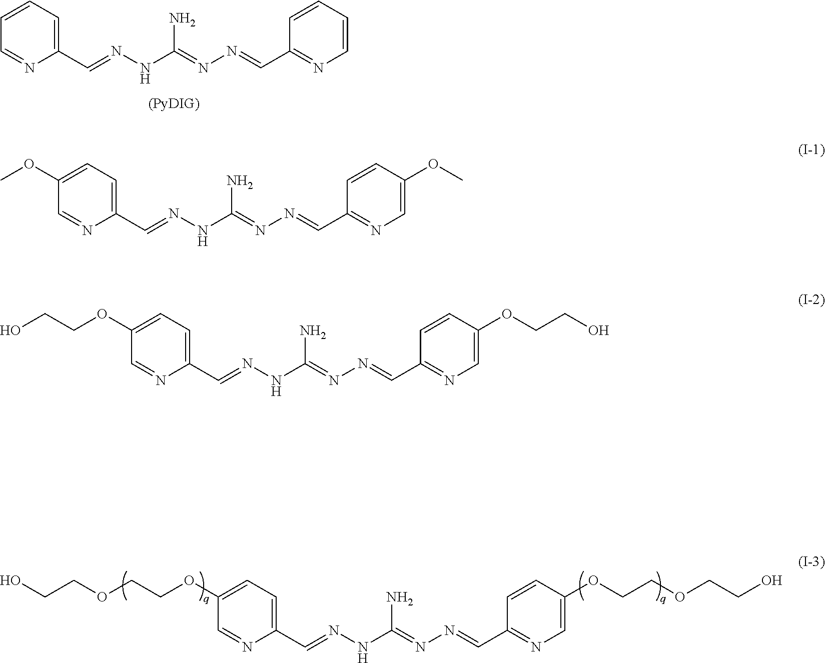

wherein q is an integer of 1-12, or more particularly, 1-8, 1-6, 1-4, 1-3, 2-12, 2-10, 2-8, 2-6, 3-12, 3-8, or 3-6. Although q values are independently selected and may be different, more typically both q values are the same.

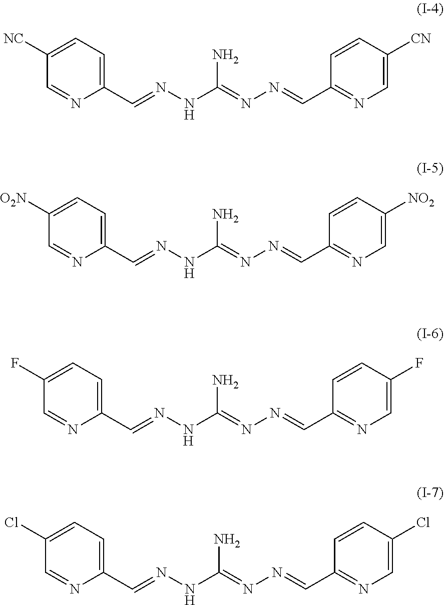 [SMILES: N#Cc1ccc(/C=N/N=C(\N)N/N=C/c2ccc(C#N)cn2)nc1 ; N/C(=N\N=C\c1ccc([N+](=O)[O-])cn1)N/N=C/c1ccc([N+](=O)[O-])cn1 ; N/C(=N\N=C\c1ccc(F)cn1)N/N=C/c1ccc(F)cn1 ; N/C(=N\N=C\c1ccc(Cl)cn1)N/N=C/c1ccc(Cl)cn1]

Moreover, although Formula (1) and sub-formulas and specific compounds thereof depict a specific tautomeric arrangement, Formula (1) is intended to include any other tautomers that can be derived from or interconvert with the tautomer shown in the formulas. As well known, tautomeric structures have the same atomic connections (aside from one or more protons) but differ in the placement of double bonds, generally with concomitant relocation of one or more protons.

The compounds according to Formula (1) can be synthesized by methods well known in the art, as further discussed in the Examples section. For example, to produce compounds according to Formula (1) in which R¹-R¹⁰ are all hydrogen atoms, 2-pyridinecarboxyaldehyde (2-formylpyridine) can be condensed with an N,N-diaminoguanidine salt (e.g., chloride salt) under moderate temperature and other conditions suitable for effecting an imine condensation. Similarly, to produce compounds according to Formula (1) in which R¹-R⁸ are hydrogen atoms and R⁹ and R¹⁰ are alkyl (e.g., methyl) groups, a 2-acylpyridine (e.g., 2-acetylpyridine) can be condensed with an N,N-diaminoguanidine salt. Similarly, to produce compounds according to Formula (1) in which at least one of R¹-R⁸ is an —OR′ group, an alkoxy- or hydroxy-substituted 2-formylpyridine or 2-acylpyridine can be condensed with an N,N-diaminoguanidine salt. In some embodiments, the imine condensation is performed in ethanol with heat. The glycol, cyano, nitro, and halogenated molecules can be made by purchasing the precursors and performing the imine condensation. An example is provided as follows:

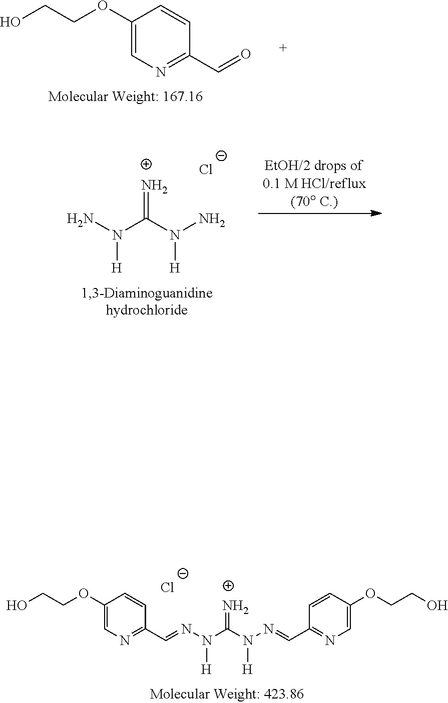 [SMILES: O=Cc1ccc(OCCO)cn1 ; [Cl-] ; NNC(=[NH2+])NN ; [Cl-] ; [NH2+]=C(N/N=C/c1ccc(OCCO)cn1)N/N=C/c1ccc(OCCO)cn1]

In another aspect, the invention is directed to an aqueous solution containing: (a) an aqueous solvent, (b) an amino acid or oligopeptide of the formula H₃N⁺-L-COO⁻, wherein L has the formula —(CH₂)ᵣ— wherein r is an integer of 1-6 with optional interruption of one or more carbon-carbon bonds with one or more —C(O)NH— linkages, and (c) a photoactive compound of the following Formula (1). As further discussed below, the aqueous solution is useful for capturing carbon dioxide (CO₂) along with a regeneration step that requires substantially less energy input than conventional thermal methods. In some embodiments, the aqueous solution may contain one or more additional components (e.g., an auxiliary agent, such as an antimicrobial agent, pH adjusting or controlling substance, or anti-foaming agent), while in other embodiments, the aqueous solution is limited to components (a), (b), and (c) and one or more auxiliary agents, or instead limited to components (a), (b), and (c).

Component (a) of the aqueous solution is the aqueous solvent. The aqueous solvent may be composed of solely (100%) water or a mixture of water and a non-reactive water-miscible solvent. In the case of a mixed aqueous solvent, the non-reactive water-miscible solvent may be, for example, a glycol of the formula —HO(CH₂CH₂O)ₚH, wherein p is an integer of 1-12 (or more particularly, 1-8, 1-6, 1-4, or 1-3); dimethylsulfoxide (DMSO); and/or dimethylformamide (DMF). In some embodiments, the aqueous solvent excludes one or more of methanol, ethanol, propanol, isopropanol, acetonitrile, acetone, carbonate solvents, ester solvents, amine solvents, or cyclic ether solvents. In the case of a mixed aqueous solvent, water may be present in an amount of 0.1-99.9 vol %, or more particularly, 1-95 vol %, 1-80 vol %, 1-50 vol %, 1-30 vol %, 10-95 vol %, 10-80 vol %, 10-50 vol %, 10-30 vol %, 20-95 vol %, 20-80 vol %, 20-50 vol %, 30-95 vol %, 30-80 vol %, 30-60 vol %, or 30-50 vol %.

Component (b) of the aqueous solution is an amino acid or oligopeptide (e.g., dipeptide, tripeptide, or tetrapeptide) of the formula H₃N⁺-L-COO⁻, wherein L has the formula —(CH₂)ᵣ— wherein r is an integer of 1-6 with optional interruption of one or more carbon-carbon bonds with one or more (e.g., 1, 2, or 3) —C(O)NH— (amide) linkages. As well known, a dipeptide contains a single amide linkage, a tripeptide contains two amide linkages, a tetrapeptide contains three amide linkages, and so on. Component (b) may also be referred to as a sorbent. In some embodiments, the amino acid or oligopeptide (or specifically, L) may exclude amine (NH₂) or sulfur-containing side chains, substituents, or linkers. The amino acid or oligopeptide (or specifically, L) may or may not include one or more hydroxy (OH) and/or carboxy (COOH) groups (i.e., in addition to the terminal carboxy group). In some embodiments, the amino acid or oligopeptide is or includes glycine (gly), N-methylglycine (sarcosine, i.e., sar), and/or alanine. Some examples of such amino acids and oligopeptides include glycine, diglycine, triglycine, alanine, dialanine, trialanine, glycine-alanine, glycine-glycine-alanine, sarcosine, disarcosine, trisarcosine, glycine-sarcosine, sarcosine-glycine, alanine-sarcosine, glycine-glycine-sarcosine, sarcosine-sarcosine-glycine, glycine-sarcosine-glycine, and glycine-alanine-sarcosine. In other embodiments, the amino acid or oligopeptide is or includes serine (ser), threonine, aspartic acid, and/or glutamic acid, any of which may or may not be combined with any of the glycine-, alanine-, or sarcosine-containing peptides provided above, e.g., serine, diserine, triserine, glycine-serine, serine-glycine, serine-sarcosine, glycine-serine-glycine, glycine-glycine-serine, serine-serine-glycine, threonine, dithreonine, trithreonine, glycine-threonine, serine-threonine, threonine-glycine, glycine-threonine-glycine, glycine-glycine-threonine, threonine-threonine-glycine, and serine-serine-threonine. In other embodiments, the amino acid or oligopeptide is or includes lysine (lys), asparagine (asn), arginine (arg), or glutamine (gln) any of which may or may not be combined with any of the glycine-, alanine-, sarcosine-, serine-, threonine-, aspartic acid- or glutamic acid-containing peptides provided above. In some embodiments, any one or more of the above types of amino acids or oligopeptides may be excluded from the aqueous solution.

Some particular examples of amino acids and oligopeptides include:

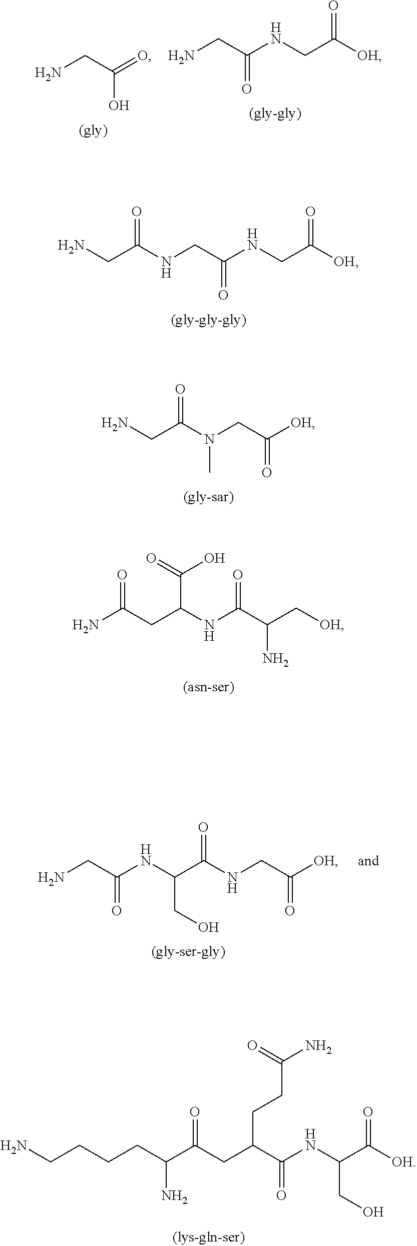

Component (c) of the aqueous solution is a photoactive compound of the Formula (1) or sub-formula thereof or specific compound thereof. Any diiminoguanidine dipyridyl compound described above within the scope of Formula (1) or sub-formula thereof can be used as the photoactive compound of component (c).

In another aspect, the present disclosure is directed to a method for capturing (and more typically, selectively capturing) carbon dioxide (CO₂) by contacting a gaseous source containing CO₂ with the above-described aqueous solution containing components (a), (b), and (c) during exposure of the aqueous solution to electromagnetic radiation (hv) having a wavelength in a range of 200-600 nm (e.g., at least 200, 250, or 300 nm, and up to or below 600, 550, 500, 450, 400, or 350 nm, e.g., 200-400 nm). The radiation functions to convert the E,E (open) configuration of compound of Formula (1) to a Z,Z (closed) configuration shown below as Formula (1′). As the Z,Z configuration is more basic than the E,E configuration and has a higher pKa than the amino acid or oligopeptide, the Z,Z configuration has an ability to abstract an ammonium hydrogen atom from the amino acid or oligopeptide, thereby converting the ammonium form of the amino acid or oligopeptide to a more reactive deprotonated amine form denoted by the formula H₂N-L-COO⁻. The foregoing reactive amine form of the amino acid or oligopeptide reacts with CO₂ to form a carbamate of the formula ⁻O(O)CN(H)-L-COO⁻. The foregoing carbamate form typically reacts with surrounding water molecules to form bicarbonate and more protonated H₃N⁺-L-COO⁻ species, which is again converted to the reactive amine form by contact with the Z,Z configuration of the photoactive compound. The photoactive compound remains in the Z,Z form as long as it is exposed to the electromagnetic radiation. When the aqueous solution is no longer exposed to the electromagnetic radiation, the Z,Z form of the photoactive compound reverts back to the E,E form along with simultaneous protonation of the bicarbonate (HCO₃⁻) anion and release of CO₂. The foregoing step of ceasing exposure to the electromagnetic radiation functions as a regeneration step of the amino acid or oligopeptide sorbent. Subsequent exposure of the aqueous solution to the radiation initiates another cycle of converting the E,E form to the Z,Z form along with capture of CO₂ as set forth above. The cycle can be repeated indefinitely. The released CO₂ can be pressurized and stored for later use in a carbon dioxide conversion process (to produce a valuable commodity chemical), oil enhanced recovery, or a dry ice manufacturing process.

The gaseous source making contact with the aqueous solution can be, for example, air, waste gas from an industrial or commercial process, flue gas from a power plant, exhaust from an engine, or sewage or landfill gas, any of which may be raw or cleansed upon contact with the aqueous solution. In some embodiments, the gaseous source contains only carbon dioxide, or carbon dioxide and water (humidity), or carbon, water, and one or more other gaseous species (e.g., nitrogen, oxygen, argon, and/or hydrogen).

The aqueous solution can include any of the above described photoactive diiminoguanidine compounds within the scope of Formula (1) or sub-formula or specific compounds thereof. More specifically, a gaseous source of carbon dioxide is contacted with the above-described aqueous solution containing any of the above described photoactive diiminoguanidine compounds within the scope of Formula (1) or sub-formula or specific compounds thereof and the amino acid or oligopeptide sorbent, while the aqueous solution is exposed to electromagnetic radiation (hv) having a wavelength in a range of 200-600 nm to convert the E,E (open) configuration of the compound of Formula (1) to the Z,Z (closed) configuration of the compound of Formula (1). The Z,Z (closed) configuration of the compound of Formula (1) is shown below as the Formula (1′).

The process can be conveniently described by the following scheme:

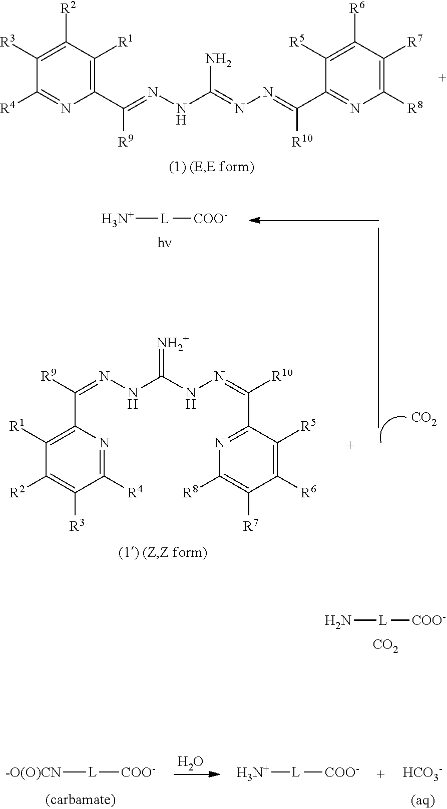

Notably, the generic Formula (1) in the above scheme may be substituted by a sub-formula of Formula (1) or any one or more specific compounds within the scope of Formula (1) as described above. Similarly, the amino acid or oligopeptide denoted as H₃N⁺-L-COO⁻ in the above scheme may be substituted by any one or more specific amino acids or oligopeptides disclosed in the present disclosure. Moreover, any one or more groupings of compounds or specific compounds within the scope of Formula (1) may be combined with any one or more specific amino acids or oligopeptides disclosed in the present disclosure.

Examples have been set forth below for the purpose of illustration and to describe certain specific embodiments of the invention. However, the scope of this invention is not to be in any way limited by the examples set forth herein.

### EXAMPLES

### Overview

The following work is directed to a photochemically-driven DAC process (photo-DAC) in which atmospheric CO₂ capture by an aqueous oligopeptide, i.e., glycylglycine (GlyGly), is achieved by subjecting a guanidine photobase, i.e., a pyridine-substituted diaminoguanidine (e.g., PyDIG), to a pH swing. The structure of PyDIG is as follows:

Upon irradiation with UV light, the PyDIG photobase undergoes photoisomerization from the E,E to the Z,Z isomer, corresponding to a pKa increase of 2.8 units that activates an amino acid or oligopeptide (e.g., GlyGly) for direct air capture (DAC) by deprotonation. After the GlyGly/PyDIG solvent is saturated with atmospheric carbon dioxide, leaving it in the dark under ambient conditions leads to isomerization of PyDIG from the Z,Z back to the E,E isomer, which is accompanied by a pH drop and CO₂ release. To demonstrate the recyclability of the GlyGly/PyDIG solvent, seven consecutive DAC cycles were completed, with a measured average cyclic capacity in the range of 0.21-0.26 mols CO₂ per mol of GlyGly/PyDIG, and minimal loss in efficiency from one cycle to another. These results open the prospect for energy-efficient DAC cycles completed entirely at ambient conditions, thereby avoiding the significant energy penalties associated with heating and boiling aqueous solvents.

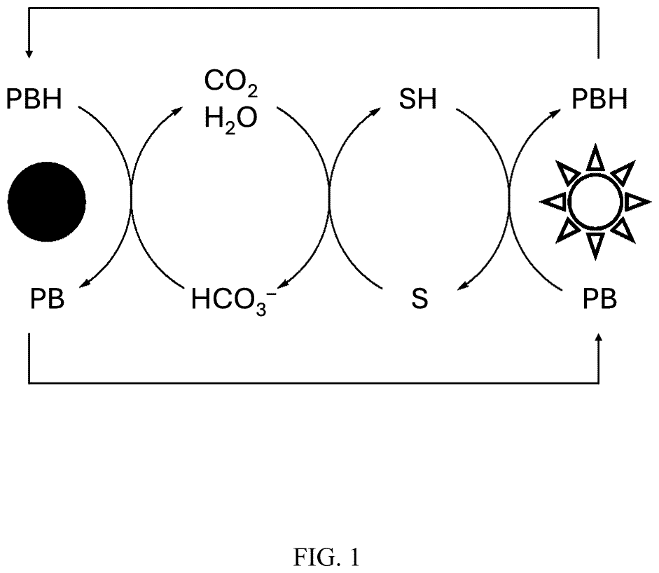

The present disclosure is directed to a more efficient approach in which a photo-controlled DAC (photo-DAC) process (using photobases) is used to achieve a pH swing required for atmospheric CO₂ binding and release. In direct contrast to photoacids, photobases become more alkaline (higher pKa of the conjugate acid) upon photoexcitation with UV or visible light. As shown schematically in FIG. 1, the corresponding increase in pH may lead to deprotonation of a sorbent, thereby activating it for atmospheric CO₂ capture. The sorbent then reacts with CO₂, converting it to bicarbonate. Once the solvent is saturated with CO₂, leaving it at room temperature in the dark allows the photobase to undergo thermal relaxation, thereby lowering the pKa of its conjugate acid, and in turn, lowering the solution pH down to the range of carbonic acid's pKa. This triggers proton transfer to bicarbonate with formation of carbonic acid, which releases the CO₂, thereby closing the DAC cycle.

### Photo-DAC Design

Implementation of the photo-DAC cycle shown in FIG. 1 requires a functional photobase (PB) and a matching sorbent (S) that satisfy several criteria. First, the selected PB needs to have a sufficiently wide pKa swing between its photoexcited and dark states, to allow CO₂ capture and release under DAC conditions. Practically, the pKa of the conjugate acid of the photoisomerized PB needs to be higher than the pKa of SH, so the PB can deprotonate the SH and achieve CO₂ capture. At the same time, the corresponding dark pKa of the PBH needs to be in the same range as that of carbonic acid (apparent pKa 6.35), so that it can protonate bicarbonate and convert it back to CO₂. Second, the lifetime of the excited or photoisomerized state PB must be sufficiently long, so that the increase in the pH of the bulk solution persists long enough to allow for S to capture the atmospheric CO₂, which is typically a slow process taking hours to days. Finally, both the PB and S need to have relatively good aqueous solubilities and stabilities in both water and air, to provide a practical DAC with a reasonable CO₂ capacity that can be sustained over multiple cycles. Notably, the photobases studied to date do not satisfy most of these criteria and are therefore inadequate for DAC.

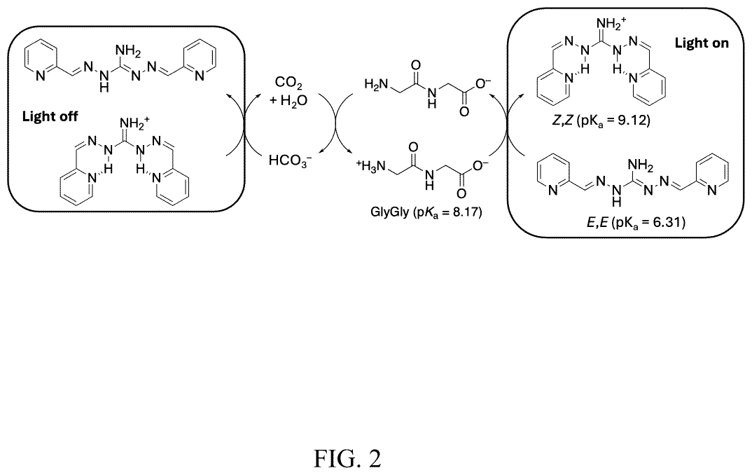

The present work utilized a novel water-soluble photobase based on a pyridine-substituted diiminoguanidine (e.g., PyDIG) that under UV irradiation undergoes photoisomerization from the E,E to the Z,Z isomer, corresponding to a pKa increase of 2.8 units that persists for days. When combined with an oligopeptide DAC sorbent, specifically glycylglycine (GlyGly), the resulting aqueous solvent can perform multiple photo-DAC cycles at ambient temperatures with an average cyclic capacity in the range of 0.21-0.26 mols CO₂ per mol of GlyGly/PyDIG. FIG. 2 schematically depicts a photo-DAC cycle using aqueous PyDIG photobase and diglycine (GlyGly) oligopeptide, as described above.

### Syntheses of Photobases

### Bis(2-((E)-pyridin-2-ylmethylene)hydrazineyl)methaniminium chloride

E,E-PyDIG·HCl was synthesized via previously described methods (R. J. Abraham et al., J. Med. Chem. 59, 2126-2138, 2016). Briefly, the condensation between 2-pyridinecarboxyaldehyde (4.40 mmol, 420 μL) and N,N′-diaminoguinidine hydrochloride (2.05 mmol, 257 mg) was performed in a 50 mL vessel in 10 mL ethanol. The reaction mixture was heated to 70° C., left to react for 24 h, then cooled to room temperature, where a yellow solid formed. Diethyl ether was added (5 mL) and the flask was chilled to further induce crystallization. The yellow precipitate was filtered and washed three times with 5 mL cold diethyl ether. The product was obtained as a yellow solid (459 mg, 74% yield). ¹H NMR (400 MHz, DMSO-d6) δ 12.31 (s, 2H), 8.75 (s, 1H), 8.67-8.66 (d, J=4.8 Hz, 2H), 8.48 (s, 2H), 8.41-8.39 (d, J=8.0 Hz, 2H), 8.01-7.97 (t, J=7.7 Hz, 2H), 7.53-7.50 (t, J=12.4 Hz, 2H). ¹³C NMR (100 MHz, DMSO-d6) δ 152.1, 149.7, 147.7, 145.5, 137.7, 125.5, 121.9; HRMS (ES+) m/z: [M+H⁺] calcd. 268.1311, found 268.1305. Elemental analysis: calcd. (found) for PyDIG·HCl·3H₂O: C, 43.64 (44.14), H, 5.63 (5.35), N, 27.40 (27.42).

### (E)-N-((E)-pyridin-2-ylmethylene)-2-(pyridin-2-ylmethylene)hydrazine-1-carboximidhydrazide (PyDIG)

E,E-PyDIG·HCl (0.86 mmol, 262 mg) was dissolved in DI water (5 mL). To the yellow solution, 1 M sodium hydroxide (0.86 mmol, 862 L) was added dropwise to produce a yellow precipitate. The yellow solid was filtered and washed three times with 5 mL cold DI water. The product was obtained as a yellow solid (232 mg, 89% yield). Single crystals were grown from a 5 mM solution of PyDIG·HCl in water, with the pH adjusted to 9 with NaOH. ¹H NMR (400 MHz, DMSO-d6) δ 11.36 (s), 8.60-8.54 (d, J=4.5, 2H), 8.33-8.26 (d, J=8.0 Hz, 2H), 8.19 (s, 2H) 7.89-7.80 (t, J=7.2 Hz, 2H), 7.46 (bs) 7.40-7.33 (t, J=11.9 Hz, 2H). ¹³C NMR (100 MHz, DMSO-d6) δ 158.1, 154.4, 149.2, 144.2, 136.4, 123.3, 120.1. Elemental analysis: calcd. (found) for PyDIG·H₂O: C, 54.73 (55.08), H, 5.30 (5.10), N, 34.37 (35.51).

### Photoirradiation Experiments

In a typical photoirradiation experiment, 20 mL of a freshly prepared aqueous solution of PyDIG solution was placed in a glass vial with a diameter of 2.8 cm, stirred, and irradiated with UV-visible light using an unfiltered commercial super high-pressure mercury lamp with emissions in the long-wave UV region and in the visible region at the yellow, green, blue, and violet wavelengths. The pH was measured using a commercial pH/ISE meter with a commercial pH electrode. ¹H NMR spectra were recorded on a 500 μL aliquots before and after irradiation using a commercial 400 MHz NMR spectrometer equipped with a 5 mm PABBO probe with the water peak suppressed after 100 μL addition of D₂O. The residual solvent signal (D₂O, δH=4.80 ppm) was considered as an internal reference to calibrate the spectra. The amounts of E,E and Z,Z isomers present during cycling experiments were determined using quantitative ¹H NMR with dimethylsulfone as internal standard (δ=3.0 ppm (s, 6H)). The spectra were baseline corrected and integrated using commercial software. The chemical shifts used to monitor the photoisomerization were the set of doublets at 8.44 and 8.64 ppm and the singlets at 8.08 and 7.53 ppm for the E,E and Z,Z isomers, respectively. Absorbance spectra in D₂O were collected using a UV-Vis-NIR absorption spectrometer in a standard quartz sample cell of 10 mm×10 mm with a 3 mL volume.

### pKa Determination of PyDIG Isomers

For absorption titration experiments, a freshly prepared stock solution of 1.0 mM E,E-PyDIG·HCl in DI H₂O was added to a 1 mm quartz cuvette, and a fresh solution of NaOH (8.0 mM) was added in 2 μL additions. The solution was mixed and absorption spectra were collected after each addition using a commercial UV-Visible spectrophotometer at 24° C. For the Z,Z-PyDIG×HCl system, the E,E-2PyDIG×HCl sample was first irradiated and the extent of photoisomerization (95.2±1.6%) was determined by NMR before the titration measurements.

### Determination of Photoisomerization Quantum Yield

A chemical actinometer based on a low concentration methanol solution of azobenzene as the standard was used for this determination (J. H. Kuhn et al., Pure and Applied Chemistry 76, 2105-2146 (2004). Specifically, the output of a 1000 W xenon arc lamp operating at 400 or 500 W was spectrally selected using a customized bandpass filter with center wavelength of 334.0+0.3/−0.1 nm, bandwidth of 1.5±0.3 nm, and transmission >20% (Andover) after passing through two spatial filters with 1″ opening. The selected portion of light was directed into a light-tight black box and loosely focused to the sample using a quartz lens. Upon fully warming up and stabilization of the lamp, light exposure time was controlled using a DC motor-driven optical shutter connected to a N-LINK Timer Outlet. Absorption spectra of the standard and sample were acquired using a commercial spectrometer. To eliminate unwanted light exposure, all ambient light was switched off during this experiment. The photon flux was measured three times using an commercial UV-A sensor (305-390 nm). The lamp was warmed up for 1 h, and the meter was placed the same distance from the source as samples during irradiation experiments (˜22 cm). The measured total flux of the light was 553.3±3.1 mol m⁻² s⁻¹.

### Photo-DAC Cycles

In a typical cycling experiment, 120 mL of a freshly prepared aqueous solution of E,E-PyDIG (1 mM) and glycylglycine (1 mM) was placed in a glass beaker with a diameter of 7 cm. The solution was stirred while being irradiated with a UV-visible light source. pH values were recorded every 30 seconds for 24 hours and ¹H NMR spectra were recorded after 24 h of irradiation. After 24 h, 9 mL of sample was removed for total inorganic carbon (TIC) analysis, and then the beaker was moved into the fume hood to allow the CO₂ to be released from solution into the air. After 5 d, 9 mL of sample was removed for TIC analysis. Degassed DI water was added to the solution as needed to compensate for water evaporation and maintain a constant volume. This process was repeated seven times.

Light-Driven pH Switching with Photobase

Although the protonated PyDIG had already been found to function as an effective photoswitchable anion receptor in DMSO, the ability of the neutral PyDIG to act as a photobase in water, and its efficacy at modulating the solution pH with light has yet to be demonstrated. To this end, a 1 mM aqueous solution of PyDIG was first irradiated with a broadband UV light for 24 h and the extent of photoisomerization by ¹H NMR spectroscopy was measure. The initial PyDIG is present primarily as the E,E isomer, mixed with a negligible amount of the Z,Z and E,Z isomers (1.1±0.8%). Upon photoirradiation, most PyDIG (92.0±2.5%) was isomerized to the Z,Z isomer. The photoisomerization reaction was accompanied by a substantial increase in the solution pH (1.7 units), from 7.89 to 9.59, while the solution temperature increased slightly from 23 to 27° C. Leaving the solution for 5 days in the dark at room temperature, the pH returned to 7.95, while ¹H NMR spectroscopy indicated that most PyDIG thermally relaxed back to the E,E isomer (85.8±2.7%).

The pH can be cycled multiple times, as demonstrated here for three consecutive light-dark cycles (Table 1, below). The magnitude of the change in pH slightly decreases with each successive photoirradiation, which can be attributed to photoswitching fatigue. This points to the need to design and study other PyDIG derivatives with improved fatigue resistance over more extended cycling.

TABLE 1
Photo-controlled pH cycling in 1 mM aqueous PyDIG.

| | | | | Relaxation time |
|---|---|---|---|---|
| Cycle | pHᵢ | pH_{f} | ΔpH | (days) |
| 1 | 7.89 ± 0.06 | 9.59 ± 0.09 | 1.70 ± 0.15 | 5 |
| 2 | 7.95 ± 0.10 | 9.56 ± 0.10 | 1.61 ± 0.20 | 6 |
| 3 | 7.96 ± 0.12 | 9.53 ± 0.13 | 1.57 ± 0.25 | 5 |

The UV-vis absorption spectra from the neutral and protonated forms of both the E,E-PyDIG and the Z,Z-PyDIG isomers display clearly distinct and intense peaks corresponding to π-π* transitions (Einkauf, J. D., et al., Chemistry—A European Journal 28, e202200719, 2022). Specifically, the protonated and the neutral forms of E,E-PyDIG absorb UV light at 312 and 351 nm, respectively, while the corresponding peaks of the protonated and the neutral forms of Z,Z-PyDIG are found at 319 and 358 nm, respectively. Thus, the pKa values for both isomers could be readily determined by spectrophotometric titrations of the corresponding chloride salts with NaOH, yielding values of 6.31 and 9.12 for the E,E and Z,Z isomers, respectively.

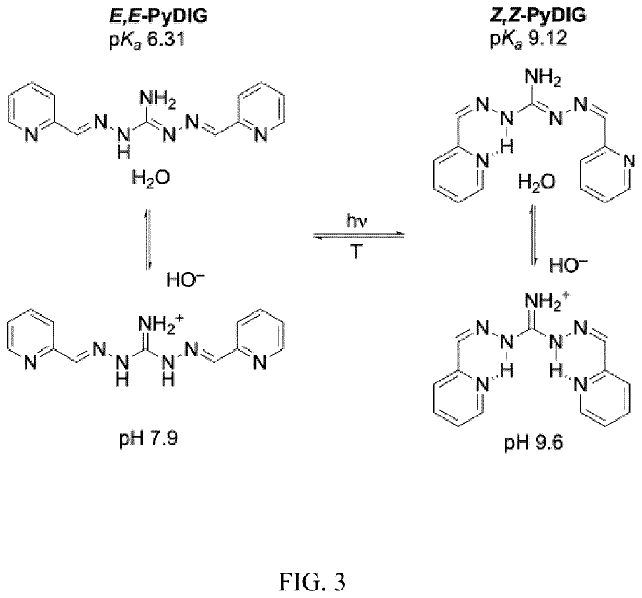

The acid-base chemistry and photochemistry of PyDIG, as surmised from the experiments described above, are summarized in FIG. 3. The diiminoguanidine group makes E,E-PyDIG a moderately strong base, with a measured pKa of 6.31. As such, when dissolved in water, it becomes partly protonated and generates a mildly alkaline solution with a pH around 7.9. Photoirradiation with UV light causes the two imine bonds of PyDIG to switch from the E to the Z configuration, a structural change that significantly destabilizes Z,Z-PyDIG relative to the starting E,E isomer. As a result, protonation of Z,Z-PyDIG becomes more favorable, leading to enhanced basicity (pKa 9.12) compared to E,E-PyDIG, and a corresponding solution pH increase to 9.6. It can be reasonably assumed that the charge-assisted intramolecular N—H . . . N hydrogen bonds kinetically stabilize the protonated Z,Z-PyDIG, thereby making thermal relaxation back to E,E-PyDIG relatively slow at ambient temperature. Nevertheless, the mixture thermally equilibrates after a few days, and the pH falls back close to the initial value. Thus, the solution pH can be cycled up and down in the presence or absence of UV light, respectively, thereby providing a photochemical means to control CO₂ binding and release at ambient conditions (vide infra).

### Light-Driven Direct Air Capture

Having established the photobase functionality of PyDIG, next we explored its ability to remove atmospheric CO₂ (Table 2, below). A 1 mM aqueous solution of PyDIG left open to air for 24 h, either in the dark or under the ambient laboratory light, absorbed negligible amounts of CO₂, as measured by total inorganic carbonate (TIC), in the range of 0.082-0.098±0.014 mols CO₂/mol PyDIG. Under UV irradiation, the amount of atmospheric CO₂ captured by PyDIG was only slightly higher: 0.131±0.009 mols CO₂/mol PyDIG. These results are sensible, considering that, like other iminoguanidines previously explored for DAC, PyDIG is not expected to react directly with CO₂; instead, its CO₂-capture functionality is primarily secured by water dissociation to generate hydroxide anions (FIG. 3), whose concentration is limited by the solution pH. At the measured pH of 9.6 for the photoirradiated PyDIG solution, the corresponding hydroxide concentration is merely 4×10⁵ M. Iminoguanidines were herein combined with amine-based sorbents, such as amino acids or oligopeptides, to greatly enhance their DAC functionality. Here, the GlyGly oligopeptide was selected as a DAC sorbent based on its pKa value of 8.17 which is in the same range as that of PyDIG.

On its own, GlyGly captures negligible amounts of atmospheric CO₂ over the course of 24 h, whether left in the dark (0.101±0.006 mols CO₂/mol GlyGly), under ambient laboratory light (0.093±0.011 mols CO₂/mol GlyGly), or under UV irradiation (0.121±0.005 mols CO₂/mol GlyGly). This is because, like amino acids, peptides exist in aqueous solutions primarily as zwitterions. Thus, their terminal amine groups are protonated and therefore not reactive toward CO₂. To activate peptides for DAC, a base sufficiently strong to deprotonate their ammonium groups is required. In the specific case of GlyGly, DAC activation requires a base with a pKa of 8.17 or greater. This condition is satisfied by Z,Z-PyDIG (pKa 9.12) but not by E,E-PyDIG (pKa 6.31). Based on this premise, it was herein hypothesized that PyDIG can be employed as a photobase that activates GlyGly for DAC by UV-light irradiation.

To test this hypothesis, aqueous solutions containing equimolar mixtures (1 mM) of PyDIG and GlyGly were left open to air for 24 h either in the dark, under ambient lab light, or irradiated with UV light. As summarized in Table 2 below, the GlyGly/PyDIG mixtures capture negligible amounts of CO2 in the dark (0.084±0.013 mM) or under ambient light (0.086±0.018), comparable to the individual GlyGly or PyDIG solutions (vide supra). In direct contrast, the GlyGly/PyDIG mixture irradiated with UV light absorbs significantly more CO₂ from the air (0.298±0.009 mM), which represents a 3.5-fold increase compared to the solutions left in the dark or under ambient lab light.

TABLE 2
Atmospheric CO2 capture by different aqueous absorbents
(1 mM) left open to air at ambient temperature for 24 h.

| Absorbent^{a} | Treatment | [CO₂]^{b} (mM) |
|---|---|---|
| PyDIG | Dark | 0.098 ± 0.014 |
| | Lab Light | 0.082 ± 0.009 |
| | UV Light^{c} | 0.131 ± 0.009 |
| GlyGly | Dark | 0.101 ± 0.006 |
| | Lab Light | 0.093 ± 0.011 |
| | UV Light^{c} | 0.121 ± 0.005 |
| GlyGly/PyDIG | Dark | 0.084 ± 0.013 |
| | Lab Light | 0.086 ± 0.018 |
| | UV Light^{c} | 0.365 ± 0.014 |
| GlyGly/KOH | Lab Light | 0.469 ± 0.006 |
| | 24 h Reflux^{d} | 0.319 ± 0.011 |
| | 48 h Reflux^{d} | 0.189 ± 0.014 |

^{a}All sorbents were tested as 1 mM aqueous solutions;
^{b}CO₂ concentration in solution, measured by TIC, after 24 h of passive exposure to air at ambient conditions;
^{c}Super high-pressure mercury lamp (254-800 nm);
^{d}Regeneration of the sorbent by heating the solution under reflux.

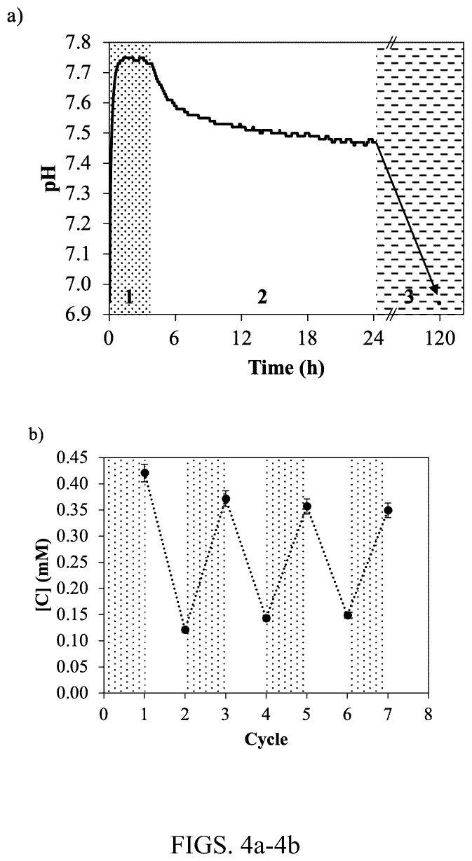

As shown in FIGS. 4a-4b, monitoring the solution pH and speciation by ¹H NMR spectroscopy provided insight into the mechanism of the photo-induced DAC. The initial GlyGly/PyDIG mixture has a pH of 6.95, a result of the two buffers present in solution: GlyGly (pKa 8.17) and E,E-PyDIG (pKa 6.31).

At this pH, the amine group of GlyGly is expected to be mostly protonated and therefore inactive for DAC. During the UV irradiation, in the first 2 hours the solution pH increases sharply by about 0.8 units, then it plateaus around 7.75 (Region 1 of FIG. 4a). This is due to the PyDIG photoisomerization from E,E to Z,Z, as evidenced by ¹H NMR spectroscopy. At this pH, which is close to the pKa of GlyGly, a significant fraction of the zwitterionic peptide is expected to be deprotonated and therefore activated for DAC. As the solution starts absorbing CO₂ from the air, the pH starts dropping (Region 2 of FIG. 4a), reaching a final value of 7.46 after 24 h. This is the result of the CO₂ reaction with GlyGly and conversion to carbamate and bicarbonate anions, as known. Finally, removing the solution from the UV-light source initiates the reverse isomerization of Z,Z-PyDIG back to E,E-PyDIG, with a residual amount of the Z,Z and E,Z isomers, as evidenced by ¹H NMR spectroscopy. The solution pH drops accordingly, returning slightly below 7.0 (Region 3 of FIG. 4a). At this pH, protonation of bicarbonate and carbamate species with the formation of the corresponding carbonic and carbamic acids become more favorable, thereby facilitating the release of CO₂ back into the air (FIG. 4b).

The GlyGly/PyDIG photo-DAC system is recyclable, as demonstrated by at least three complete CO₂ loading/release cycles with only a slight loss in efficiency (Table 3 below, FIG. 4b). The yield of PyDIG photoisomerization to Z,Z after 24 h averaged 88%, leading to an average CO₂ loading of 0.37±0.03 mols CO₂/mol GlyGly. Monitoring the DAC cycles by ¹H NMR showed that all reactions were reversible and the photoirradiation process does not seem to produce irreversible side photoreactions that can affect the DAC process. Furthermore, when the photoirradiation time was reduced to 2 h, there was no change in the overall efficiency of the process. The cyclic CO₂ capacity over three consecutive cycles, measured as the difference in the CO₂ concentration between the loaded and unloaded GlyGly/PyDIG solutions, was in the range of 0.21-0.26 mols CO₂ per mol of GlyGly/PyDIG. The long relaxation time can be shortened to 2 days by heating the solution at 80° C.; however, such extensive heating of aqueous solutions is expected to lead to high energy penalties that should be avoided in a DAC process to the extent possible.

TABLE 3
Consecutive DAC cycles with 1 mM aqueous GlyGly/PyDIG.
Each cycle consists of photoswitching of PyDIG under UV
irradiation, atmospheric CO₂ loading for 24 h, and
CO₂ release at room temperature for 5 days.

| | | | % E,E- | |
|---|---|---|---|---|
| Cycle | State | pH^{a} | PyDIG^{b} | [CO₂] (mM)^{c} |
| 1 | Before irradiation | 6.88 ± 0.02 | 97.9 ± 1.1 | 0.097 ± 0.002 |
| | After irradiation/CO₂ | 7.81 ± 0.04, | 2.2 ± 3.6 | 0.389 ± 0.031 |
| | capture | 7.57 ± 0.07 | | |
| 2 | Before irradiation | 6.99 ± 0.02 | 92.1 ± 2.9 | 0.133 ± 0.012 |
| | After irradiation/CO₂ | 7.71 ± 0.04, | 16.1 ± 5.4 | 0.365 ± 0.010 |
| | capture | 7.44 ± 0.07 | | |
| 3 | Before irradiation | 7.03 ± 0.05 | 92.8 ± 3.5 | 0.142 ± 0.001 |
| | After irradiation/CO₂ | 7.68 ± 0.07, | 19.2 ± 6.9 | 0.358 ± 0.004 |
| | capture | 7.46 ± 0.12 | | |
| 4 | Before irradiation | 7.03 ± 0.04 | 92.1 ± 4.3 | 0.145 ± 0.005 |
| | After irradiation/CO₂ | 7.62 ± 0.05, | 16.7 ± 5.8 | 0.357 ± 0.010 |
| | capture | 7.41 ± 0.16 | | |

^{a}When two pH values are listed, the first one indicates the highest pH reached during photoirradiation, while the second one is the equilibrium pH after atmospheric CO₂ capture;
^{b}Mol % determined by ¹H NMR spectroscopy;
^{c}CO₂ concentration in solution determined by TIC and is an average of three experiments.

The GlyGly/PyDIG photo-DAC system was benchmarked against aqueous GlyGly/KOH. In this conventional DAC system, the strong base KOH completely deprotonates GlyGly, activating it for reaction with CO₂ and leading to a relatively high atmospheric CO₂ loading of 0.469±0.006 mM (Table 2). However, in the absence of photoactivity, thermal regeneration of this absorbent requires prolonged refluxing times. The measured cyclic CO₂ capacity after 24 h of refluxing is only 0.15 mols CO₂/mol GlyGly. While the cyclic capacity increases to 0.28 mols CO₂/mol GlyGly after 48 h of refluxing, which is comparable with the value observed for the photo-DAC system, this comes at the expense of very high energy wasted on heating and boiling off water.

### CONCLUSIONS

The present work has demonstrated an aqueous DAC cycle operated entirely at ambient temperature. The GlyGly peptide is activated for DAC by PyDIG, a novel photobase that undergoes photoinduced structural changes when irradiated, increasing its pKa by 2.8 units and raising the solution pH by up to 1.7 units. Once the GlyGly/PyDIG sorbent is loaded with atmospheric CO₂, the greenhouse gas can be released by simply leaving the solution in the dark at room temperature. Thus, this novel photo-DAC approach has the potential for significant energy savings once implemented into a DAC technology.

However, the efficiency of the GlyGly/PyDIG photo-DAC system ultimately depends on several factors. First, the efficiency of the photoisomerization reaction depends on PyDIG's molar absorptivity, photon flux of the light source, and the photoisomerization quantum yield. The measured photoisomerization quantum yield of PyDIG (ΦE,E→Z,Z) is 4.01% and the light flux of excitation source is 553.3±3.1 mol m⁻² s⁻¹. Based on the UV absorption maxima of the protonated and neutral PyDIG at 312 and 351 nm, respectively, the theoretical minimum energy required in the range of 338 to 386 kJ/mol CO or 7.7 to 8.8 GJ/ton CO₂ can be estimated. Taking into account the efficiency of the photoisomerization process (quantum yield, light flux, efficiency of the UV lamp, etc.), the actual energy required will be even higher.

The above reinforces the critical need to design next generation photobases that absorb visible light, so the photo-DAC system can be fueled by abundant and renewable solar power. This was achieved with the second-generation PyDIG compounds substituted with —NO₂ or —CN groups, as shown in structures (I-4) and (I-5) above. Second, the CO₂ capacity of the solvent depends on the aqueous solubility of the photobase. Although the aqueous solubility of PyDIG, in mM range, was sufficient for the proof of concept, significant solubility improvements are needed for a practical DAC technology. This was achieved with the second-generation PyDIG compounds substituted with water-solubilizing groups, such as shown in structures (I-2) and (I-3) above. Third, the slow decrease in uptake capacity with each cycle, attributed here to photoswitching fatigue, must be addressed. Along this line, appending electron-rich substituents and creating a push-pull mechanism, as demonstrated with azobenzenes, may reduce the photoswitching fatigue observed with PyDIG. Fourth, the efficiency of the atmospheric CO₂ capture depends on the available air/liquid contact area. While in the current proof of concept this area is very limited, resulting in slow CO₂ loading into solution (24 h), significantly faster CO₂ absorption can be achieved with the use of an effective air-liquid contactor. For efficient photo-DAC technologies, in addition to high surface area, the contactor will also need to be transparent to UV and visible light to allow for sorbent photoactivation.

Finally, unlike in the current study, where the CO₂ was released back into the air, in an actual photo-DAC technology the CO₂ will have to be collected in high purity so it can be sent to long-term storage or utilization. Thus, the CO₂ release will have to be done under a CO₂ atmosphere, which will lead to a reduced driving force and the possible need for application of mild heating or vacuum.

While there have been shown and described what are at present considered the preferred embodiments of the invention, those skilled in the art may make various changes and modifications which remain within the scope of the invention defined by the appended claims.

What is claimed is:

1. A compound having the following structure: 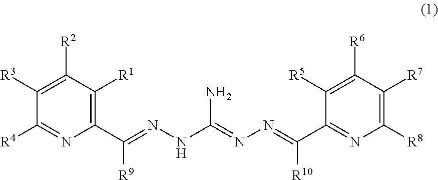
wherein:
R¹, R², R³, R⁴, R⁵, R⁶, R⁷, R⁸, R⁹, and R¹⁰ are independently selected from (i) hydrogen atom (H), (ii) alkyl groups (R) containing 1-3 carbon atoms and optionally substituted with one or more fluorine atoms, (iii) —(CH₂)ₙ(OCH₂CH₂)ₘX groups, (iv) OR′ groups, (v) NR′₂ groups, (vi) NHC(O)R′, (vii) C(O)R′ groups, (viii) halogen atoms, (ix) NO₂ groups, and (x) CN groups, wherein R′ groups are independently selected from hydrogen atom (H); alkyl groups (R) containing 1-3 carbon atoms; and groups of the formula —(CH₂)ₙ(OCH₂CH₂)ₘX, wherein n is 0 or an integer of 1-3, m is 0 or an integer of 1-12, and X is H, OH, OCH₃, or OCH₂CH₃;
provided that at least one of R¹, R², R³, R⁴, R⁵, R⁶, R⁷, R⁸, R⁹, and R¹⁰ is independently selected from NO₂, CN, —(CH₂)ₙ(OCH₂CH₂)ₘX, OR′, NR′₂, NHC(O)R′, and C(O)R′, wherein at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′ and NR′₂; and if m is 0, then X is OH, OCH₃, or OCH₂CH₃.

2. The compound of claim 1, wherein R⁹ and R¹⁰ are hydrogen atoms.

3. The compound of claim 1, wherein at least one of R¹, R², R³, R⁴, R⁵, R⁶, R⁷, and R⁸ is independently selected from NO₂, CN, —(CH₂)ₙ(OCH₂CH₂)ₘX, OR′, NR′₂, NHC(O)R′, and C(O)R′, wherein at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′ and NR′₂; and if m is 0, then X is OH, OCH₃, or OCH₂CH₃.

4. The compound of claim 1, wherein at least one of R², R³, R⁶, and R⁷ is independently selected from NO₂, CN, —(CH₂)ₙ(OCH₂CH₂)ₘX, OR′, NR′₂, NHC(O)R′, and C(O)R′, wherein at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′ and NR′₂; and if m is 0, then X is OH, OCH₃, or OCH₂CH₃.

5. The compound of claim 1, wherein at least one of R¹, R², R³, R⁴, R⁵, R⁶, R⁷, and R⁸ is NO₂ or CN.

6. The compound of claim 1, wherein at least one of R², R³, R⁶, and R⁷ is NO₂ or CN.

7. The compound of claim 1, wherein at least one of R¹, R², R³, R⁴, R⁵, R⁶, R⁷, and R⁸ is —(CH₂)ₙ(OCH₂CH₂)ₘX, or an OR′ group, wherein R′ is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′; and if m is 0, then X is OH, OCH₃, or OCH₂CH₃.

8. The compound of claim 1, wherein at least one of R², R³, R⁶, and R⁷ is —(CH₂)ₙ(OCH₂CH₂)ₘX, or an OR′ group, wherein R′ is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′; and if m is 0, then X is OH, OCH₃, or OCH₂CH₃.

9. The compound of claim 1, wherein the compound has the following structure: 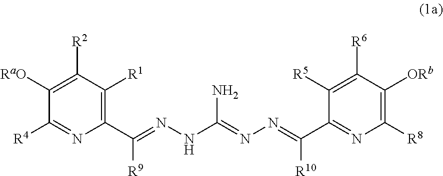
wherein at least one R¹, R², R⁴, R⁵, R⁶, R⁸, R⁹, and R¹⁰ are as defined in claim 1, and R^{a} and R^{b} are selected from —(CH₂)ₙ(OCH₂CH₂)ₘX, wherein n is an integer of 1-3, m is 0 or an integer of 1-12, and X is H, OH, OCH₃, or OCH₂CH₃, provided that if m is 0, then X is OH, OCH₃, or OCH₂CH₃.

10. A method for selectively capturing carbon dioxide (CO₂), the method comprising:
(i) providing an aqueous solution containing (a) an aqueous solvent, (b) an amino acid or oligopeptide of the formula H₃N⁺-L-COO⁻, wherein L has the formula —(CH₂)ᵣ— wherein r is an integer of 1-6 with optional interruption of one or more carbon-carbon bonds with one or more —C(O)NH— linkages, and (c) a photoactive compound of the following Formula (1): 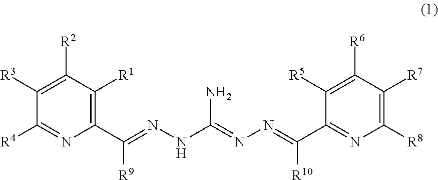
wherein:
R¹, R², R³, R⁴, R⁵, R⁶, R⁷, R⁸, R⁹, and R¹⁰ are independently selected from (i) hydrogen atom (H), (ii) alkyl groups (R) containing 1-3 carbon atoms and optionally substituted with one or more fluorine atoms, (iii) —(CH₂)ₙ(OCH₂CH₂)ₘX groups, (iv) OR′ groups, (v) NR′₂ groups, (vi) NHC(O)R′, (vii) C(O)R′ groups, (viii) halogen atoms, (ix) NO₂ groups, and (x) CN groups, wherein R′ groups are independently selected from hydrogen atom (H); alkyl groups (R) containing 1-3 carbon atoms; and groups of the formula —(CH₂)ₙ(OCH₂CH₂)ₘX, wherein n is 0 or an integer of 1-3, m is 0 or an integer of 1-12, and X is H, OH, OCH₃, or OCH₂CH₃; provided that n and m are not both 0; n is not 0 for R′ in OR′ and NR′₂; and if m is 0, then X is OH, OCH₃, or OCH₂CH₃.
(ii) contacting a source of CO₂ with the aqueous solution during exposure of the aqueous solution to electromagnetic radiation (hv) having a wavelength in a range of 200-600 nm to convert the E,E (open) configuration of compound of Formula (1) to a Z,Z (closed) configuration of Formula (1′), wherein the Z,Z configuration is more basic than the E,E configuration and has a higher pKa than the amino acid or oligopeptide in order to abstract an ammonium hydrogen atom from the amino acid or oligopeptide, wherein the deprotonated amino acid or oligopeptide captures carbon dioxide, in accordance with the following reaction process: 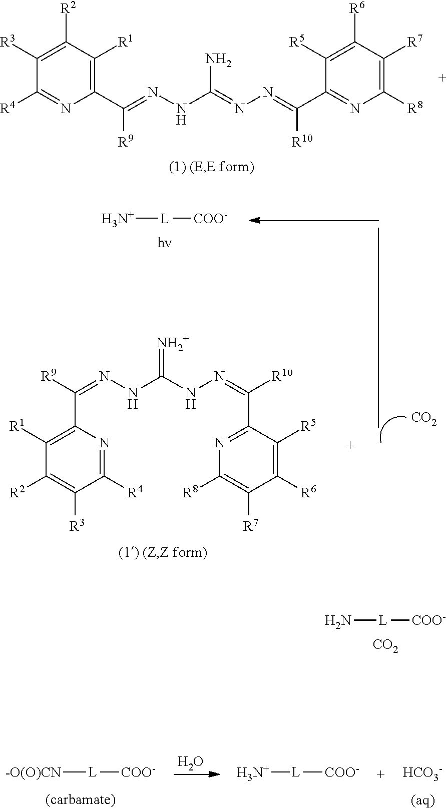
and
(iii) ceasing exposure to said electromagnetic radiation which results in conversion of the Z,Z form back to the E,E form with simultaneous protonation of the bicarbonate (HCO₃⁻) anion and release of CO₂.

11. The method of claim 10, wherein the electromagnetic radiation has a wavelength in a range of 200-400 nm.

12. The method of claim 10, provided that at least one of R¹, R², R³, R⁴, R⁵, R⁶, R⁷, R⁸, R⁹, and R¹⁰ is independently selected from NO₂, CN, —(CH₂)ₙ(OCH₂CH₂)ₘX, OR′, NR′₂, NHC(O)R′, and C(O)R′, wherein at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′ and NR′₂; and if m is 0, then X is OH, OCH₃, or OCH₂CH₃.

13. The method of claim 10, wherein R⁹ and R¹⁰ are hydrogen atoms.

14. The method of claim 10, wherein at least one of R¹, R², R³, R⁴, R⁵, R⁶, R⁷, and R⁸ is independently selected from NO₂, CN, —(CH₂)ₙ(OCH₂CH₂)ₘX, OR′, NR′₂, NHC(O)R′, and C(O)R′, wherein at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′ and NR′₂; and if m is 0, then X is OH, OCH₃, or OCH₂CH₃.

15. The method of claim 10, wherein at least one of R², R³, R⁶, and R⁷ is independently selected from NO₂, CN, —(CH₂)ₙ(OCH₂CH₂)ₘX, OR′, NR′₂, NHC(O)R′, and C(O)R′, wherein at least one R′ in any of the foregoing groups is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′ and NR′₂; and if m is 0, then X is OH, OCH₃, or OCH₂CH₃.

16. The method of claim 10, wherein at least one of R¹, R², R³, R⁴, R⁵, R⁶, R⁷, and R⁸ is NO₂ or CN.

17. The method of claim 10, wherein at least one of R², R³, R⁶, and R⁷ is NO₂ or CN.

18. The method of claim 10, wherein at least one of R¹, R², R³, R⁴, R⁵, R⁶, R⁷, and R⁸ is —(CH₂)ₙ(OCH₂CH₂)ₘX, or an OR′ group, wherein R′ is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′; and if m is 0, then X is OH, OCH₃, or OCH₂CH₃.

19. The method of claim 10, wherein at least one of R², R³, R⁶, and R⁷ is —(CH₂)ₙ(OCH₂CH₂)ₘX, or an OR′ group, wherein R′ is a group of the formula —(CH₂)ₙ(OCH₂CH₂)ₘX, wherein n is 0 or an integer of 1-3, m is 0 or an integer of 1-12, and X is H, OH, OCH₃, or OCH₂CH₃, provided that: n and m are not both 0; n is not 0 for R′ in OR′; and if m is 0, then X is OH, OCH₃, or OCH₂CH₃.

20. The method of claim 10, wherein the compound of Formula (1) has the following structure: 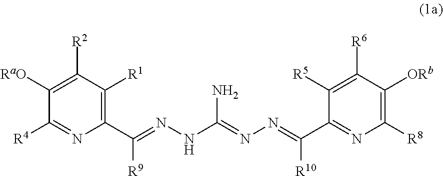
wherein at least one R¹, R², R⁴, R⁵, R⁶, R⁸, R⁹, and R¹⁰ are as defined in claim 1, and R^{a} and R^{b} are selected from —(CH₂)ₙ(OCH₂CH₂)ₘX, wherein n is an integer of 1-3, m is 0 or an integer of 1-12, and X is H, OH, OCH₃, or OCH₂CH₃, provided that if m is 0, then X is OH, OCH₃, or OCH₂CH₃.

21. The method of claim 10, wherein said aqueous solvent is a mixture of water and at least one co-solvent selected from the group consisting of: a glycol of the formula —HO(CH₂CH₂O)ₚH, wherein p is an integer of 1-12; dimethylsulfoxide (DMSO); and
dimethylformamide (DMF), wherein said water may be present in the aqueous solvent in an amount of 0.1-99.9 vol %.
